(19) United States
(12) Patent Application Publication
(10) Pub. No.: US 2005/0152891 A1
(43) Pub. Date: Jul. 14, 2005

(54) Carbon nanotubules for storage of nitric oxide

(75) Inventors: Eric J. Toone, Durham, NC (US); Jonathan S. Stamler, Chapel Hill, NC (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Duke University, Durham, NC (US)

(21) Appl. No.: 10/980,616

(22) Filed: Nov. 3, 2004

Related U.S. Application Data
(63) Continuation of application No. PCT/US03/13289, filed on May 1, 2003.
(60) Provisional application No. 60/377,862, filed on May 3, 2002.

(51) Int. Cl. A61K 33/44

(52) U.S. Cl. 424/125; 423/445 B

(57) ABSTRACT

Delivering nitric oxide to a treatment site, such as in the area of an implanted stent, over a period of hours or days is desirable; however, the storage and release of nitric oxide in medically-relevant situations and amounts is a challenge, in part due to the gaseous nature of nitric oxide and its instability in the presence of oxygen. The present invention provides a method of preparing compositions of matter, particularly those comprising nanotubules, containing nitric oxide or gases with nitric oxide-like biological activity, where the gas is non-covalently bound to the composition. These compositions allow for the storage of nitric oxide or a related gas, followed by controlled release of the gas. Compositions disclosed in the present invention include polymers, articles, pills, capsules, and medical devices.

### RELATED APPLICATIONS

This application is a continuation of International Application No. PCT/US03/13289, filed May 1, 2003, which designated the United States and was published in English, and which claims the benefit of U.S. Provisional Application No. 60/377,862, filed May 3, 2002. The entire teachings of the above applications are incorporated herein by reference.

### BACKGROUND OF THE INVENTION

Nitric oxide is a small, gaseous molecule produced endogenously by both plants and animals. In animals, nitric oxide has particularly important effects in the circulatory, immune, and nervous systems. The effects on the circulatory system include regulation of blood pressure through relaxation of the smooth muscle walls of blood vessels and prevention of clotting by inhibiting the aggregation of platelets. The release of nitric oxide in close proximity to a medical device such as a stent or an artificial heart is expected to reduce the clotting encountered with these devices, thereby reducing morbidity and mortality.

Several difficulties have been encountered in storing nitric oxide in a discrete source and delivering nitric oxide to a treatment site over a period of days or weeks. For example, nitric oxide has a short half-life, on the order of seconds, in oxygenated milieu, particularly biological milieu. Also, as a gas, nitric oxide tends to rapidly diffuse away from point sources, preventing it from being efficiently stored.

In the place of nitric oxide, various compounds, which are relatively stable in the presence of oxygen, have been used. These compounds release nitric oxide or molecules with nitric oxide-like activity upon exposure to acids, bases, metal ions, light, heat, and the like. Nitric oxide-releasing compounds include S-nitrosothiols, diazeniumdiolates (NONOates), organic nitrites, organic nitrates (e.g., nitroglycerin), metal nitrosyls (e.g., sodium nitroprusside), and nitrosylated proteins and peptides. These all represent effective sources of nitric oxide, however, the nitric oxide activity relies on a reaction to convert the above sources into nitric oxide. It is desirable to have an authentic source of nitric oxide that does not necessarily rely on the presence of enzymes, metal ions, or free thiols to convert a precursor molecule into nitric oxide.

It is therefore desirable to develop a device or composition for storing nitric oxide or a gas with nitric oxide-like activity, which allows for storage and prolonged release of the gas and does not involve covalently bonding nitric oxide or a related gas to the device.

### SUMMARY OF THE INVENTION

It has now been found that nitric oxide can be contained in hydrophobic materials, particularly nanotubules, such that nitric oxide can be stored by a hydrophobic material. It has also been found that nitric oxide can be slowly released by such hydrophobic materials over extended periods of time. For example, carbon nanotubules loaded with nitric oxide released nitric oxide continuously for over a day (Example 3), even when the nanotubule was entrained in a styrene-isobutylene copolymer (Example 5). In addition, the nitric oxide released from these nanotubules retains its biological activity. For example, rabbit aortal rings relaxed when exposed to nitric oxide-loaded carbon nanotubules (Example 2). Based on these discoveries, novel nitric oxide-containing nanotubules and methods of preparing and using such nanotubules are disclosed herein.

In one embodiment, the present invention is a composition comprising a compound that non-covalently binds nitric oxide or a gas with nitric oxide-like biological activity. Nitric oxide or a gas with nitric oxide-like biological activity is non-covalently bound to said compound. Suitable compositions include surfactants, perfluorocarbons, polythiolated cyclodextrins, and cyclodextrins.

The present invention includes a nanotubule, where the nanotubule contains nitric oxide or a gas with nitric oxide-like biological activity. The interior of the nanotubule is substantially free of oxygen.

The present invention also includes an article comprising one or more nanotubules, which each contain nitric oxide or a gas with nitric oxide-like biological activity.

In another embodiment, the present invention is a method of administering nitric oxide or a gas with nitric oxide-like properties to an individual, comprising the step of contacting an aqueous solution with an article of the present invention and administering the aqueous solution to the individual. Articles, which can be advantageously used in this method, include bags containing intravenous fluid, syringes, and medical tubing.

The present invention is also a polymer entrained with nanotubules, where the nanotubules contain nitric oxide or a gas with nitric oxide-like biological properties.

The present invention includes a method of delivering nitric oxide to a treatment site by implanting a medical device comprising one or more nanotubules containing nitric oxide or a gas with nitric oxide-like biological activity.

In another embodiment, the present invention is a method of preparing nanotubules comprising nitric oxide or a gas with nitric oxide-like biological activity. The method comprises the step of contacting the nanotubules with nitric oxide or a gas with nitric oxide-like biological activity, where the nitric oxide or the gas with nitric oxide-like biological activity is substantially free of oxygen.

The present invention has many advantages. Compositions of the present invention have the ability to store therapeutically relevant quantities of nitric oxide or related gases in an uncomplexed form. These compositions also have the ability to release stored nitric oxide in a controlled fashion, thereby serving as a long-acting source of nitric oxide. These compositions are easily prepared, by contacting a material with nitric oxide or a gas with nitric oxide-like biological activity under pressures at or exceeding ambient pressure.

### BRIEF DESCRIPTION OF THE DRAWINGS

FIG. 1 is a graph showing the release of nitrogen oxides (Nₒₓ) from nanotubules loaded with nitric oxide into phosphate-buffered saline (PBS) at 37° C.

FIG. 2 is a graph showing the release of nitrogen oxides (Nₒₓ) from nanotubules loaded with nitric oxide entrained in a styrene-isobutylene-styrene copolymer (SIBS) into phosphate-buffered saline at 37° C.

### DETAILED DESCRIPTION OF THE INVENTION

Compositions of the present invention comprise compounds which bind nitric oxide or a gas with nitric-oxide like properties non-covalently. Although Applicants do not wish to be bound by any particular mechanism, it is believed that the binding results from pi stacking, van der Waals forces, and/or hydrophobic interactions. Typically, such compositions and compounds are hydrophobic. One example of a composition of the present invention is a nanotubule containing nitric oxide or a gas with nitric oxide-like biological activity. Other compositions capable of non-covalently binding nitric oxide or a gas with nitric oxide-like properties include surfactants, perfluorocarbons, polythiolated cyclodextrins, and cyclodextrins. Another example is a composition comprising a polymer and nanotubules entrained in the polymer, where the nanotubules contain nitric oxide or a gas with nitric oxide-like biological activity.

Nanotubules of the present invention can be characterized as long symmetrical carbon tubes, which are formed from hexagonal and pentagonal graphite molecules joined at their edges. Nanotubules can additionally comprise heteroatoms or metals. Nanotubules typically have diameters of about 1 nm to about 50 nm, about 2 nm to about 25 nm, or about 5 nm to about 10 nm. Nanotubules typically have lengths of about 10 nm to about 100 μm, about 100 nm to about 10 μm, or about 500 nm to about 2 μm. Nanotubules of the present invention can be single-walled or multi-walled, where one or more single-walled nanotubules are contained within a nanotubules of greater diameter and equal or greater length. Nanotubules can have “zigzag”, armchair, helical, spiral, twisted, and untwisted shapes and geometries. Nanotubules of these types are known in the art and are disclosed, for example, in M. S. Dresselhaus, G. Dresselhaus, P. C. Eklund, “Fullerenes,” J. Mater. Res., 8(8), 2054-2097, (1993); P. E. Ross, “Buckytubes,” Sci. Am., 24, (December 1991); B. I. Yakobson and R. Smalley, “Fullerene Nanotubes: C1,000,000 and Beyond,” Am. Sci., 85(4), 324-337 (1997); J. Bernholc, C. Roland, and B. I. Yakobson, “Nanotubes,” Curr. Opin. Solid State Mater. Sci., 2, 706-715 (1997), the entire teachings of which are incorporated herein by reference.

Nanotubules of the present invention contain nitric oxide or a gas with nitric oxide-like biological activity. Preferably, nanotubules of the present invention contain nitric oxide. Typically, nitric oxide or the gas with nitric oxide-like activity contained by the nanotubule comprises about 0.5 weight percent to about 10 weight percent, about 0.5 to about 6 weight percent, about 0.5 to about 4 weight percent, or about 1 to about 3 weight percent of the nanotubule. Gases with nitric oxide-like biological activity include nitrogen dioxide, dinitrogen trioxide, and alkyl nitrites. Alkyl nitrites include ethyl nitrite, propyl nitrite, n-butyl nitrite, iso-butyl nitrite, amyl nitrite, and iso-amyl nitrite.

As defined herein, nitric oxide or a gas with nitric oxide-like biological activity is “contained” in a nanotubule when it is in the interior of such nanotubules or adsorbed on the interior or exterior surface of such nanotubules.

The interiors of nanotubules of the present invention are typically substantially free of oxygen. “Substantially free of oxygen,” as defined herein, means the interior of a nanotubule contains less than 5% oxygen by volume, preferably containing less than 2% oxygen by volume, even more preferably contains less than 1% oxygen by volume, and most preferably contains no oxygen.

Nanotubules of the present invention can optionally be functionalized with one or more functional groups on either the sides or the ends of a nanotubule. Optionally, 0 to 50% of the carbon atoms of a nanotubule can be functionalized. A wide variety of reactive groups can serve as functional groups, including those comprising nitrogen, oxygen, sulfur, phosphorus, and halides, particularly fluoride. In one example, the sides of a nanotubule are fluorinated by reacting a nanotubule with elemental fluorine. In another example, the ends of a nanotubule are functionalized with carboxylic acid or carboxylate groups. Functional groups (or functionalized nanotubules) can undergo further reaction, for example, a fluorinated nanotubule (e.g., one containing C—F bonds) can be reacted with an alkoxide, an alkyllithium complex, or a Grignard reagent (an alkylmagnesium bromide) to form an alkoxylated or an alkylated nanotubule. Typically, a functional group will not decrease the nitric oxide content (e.g. measured by weight percent nitric oxide) of a nanotubule more than two-fold, and preferably increases the nitric oxide content of a nanotubule. Functionalized nanotubules of these types are known in the art and are disclosed, for example, in E. T. Mickelson, I. W. Chiang, J. L. Zimmerman, P. J. Boul, J. Lozano, J. Liu, R. E. Smalley, R. H. Hauge, J. L. Margrave, J. Phys. Chem., 103, 4318-4322 (1999) and P. J. Boul, J. Liu, E. T. Mickelson, C. B. Huffman, L. M. Ericson, I. W. Chiang, K. A. Smith, D. T. Colbert, R. H. Hauge, J. L. Margrave, R. E. Smalley, Chem. Phys. Lett. 310, 367-372 (1999), the entire teachings of which are incorporated herein by reference.

Nanotubules of the present invention can be “capped”, “open-ended”, or “closed”. “Open-ended” nanotubules have no carbon atoms or functional groups closing off either end of the nanotubule, such that a gas, molecule, or other substance having a diameter less than that of the nanotubule can freely pass from the exterior to the interior of the nanotubule through an end of the nanotubule. Although open-ended nanotubules do not have functional groups closing off an end of the nanotubule, open-ended nanotubules typically have functional groups, such as carboxylate groups, at the ends of the nanotubule. “Closed” nanotubules have graphitic hemispheres at each end of the nanotubule. “Capped” nanotubules are partially or completely closed at one or both ends of the nanotubule by addition of a capping molecule to the end of a nanotubule, such that a gas, molecule, or other substance having a diameter less than that of the nanotubule cannot freely pass from the exterior to the interior of the nanotubule through an end of the nanotubule, and vice versa. A substance, typically in the gaseous state, having a diameter less than that of the nanotubule can more freely pass from the exterior to the interior of the nanotubule (or vice versa) through an end of the nanotubule once the capping molecule has been cleaved from the end of a molecule, such as by hydrolysis.

Advantageously, the groups which cap the end of a nanotubule are selected so that they are cleavable. Cleavable functional groups include amides, esters, carbonates, carbamates, ureas, acylureas, phosphate esters, phosphonate esters, sulfonate esters, and sulfate esters. Cleavable functional groups are generally reactive in a biological milieu and cleave on a time course relevant to release of nitric oxide or a gas nitric oxide-like activity. Cleavable functional groups are often chosen towards a utility, such that a cleavable functional group intended for pharmaceutical purposes cleaves at a target site.

Typically, a capping molecule is attached to a nanotubule through two functional groups, such that the molecule connects two carbon atoms on the end of a nanotubule. One example of a capped nanotubule of this type is represented schematically below: 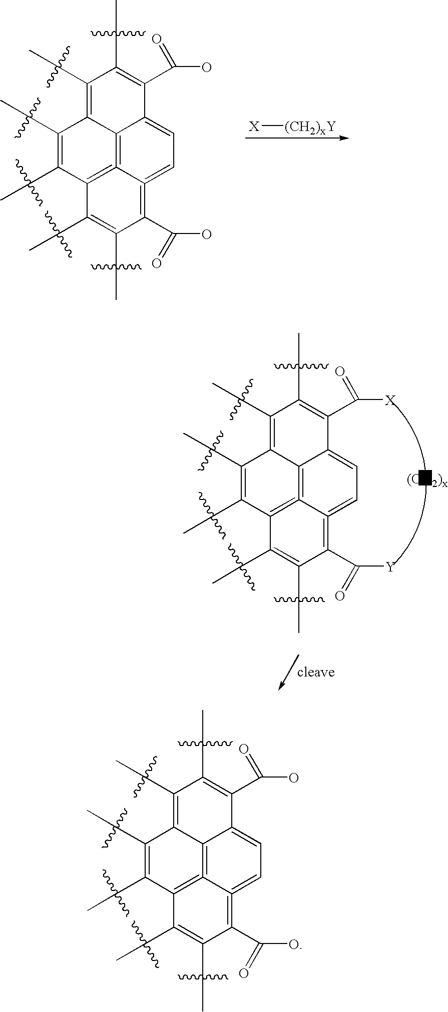
The diagram represents a portion at the end of a nanotubule, however, not all bonds are shown. As discussed above, an open-ended nanotubule can have functional groups at its ends. The nanotubule can be capped, for example, by a suitable mono- or difunctional capping reagent. A preferred difunctional reagent is an α,ω-substituted alkyl group (e.g., a C1-C24 alkyl group), which is substituted at one terminus with functional group X and at the other terminus with functional group Y, each of which can react with the functional group(s) at the end of the nanotubule. This is shown schematically above where the functional groups at the end of the nanotubule are carboxylate groups. One skilled in the art can select appropriate combinations of nanotubule functional group and capping reagent; for example, a carboxylate nanotubule functional group is reacted with a capping reagent having amino and/or hydroxyl groups. By connecting two points on the end of a nanotubule, the capping molecule more effectively limits gas exchange between the ambient atmosphere and the interior of the nanotubule. A similar effect is obtained when a monofunctional molecule serves as a capping group. Specific examples of the preparation of nanotubules with capping molecules can be found in Chen, J.; Hamon, M. A.; Hu, H.; Chen, Y.; Rao, A. M.; Eklund, P. C.; Haddon, R. C., Science, 282, 95 (1998); Wong, S. S.; Joselevich, E.; Woolley, A. T.; Cheung, C. L.; Lieber, C. M., Nature, 394, 52 (1998); Wong, S. S.; Woolley, A. T.; Joselevich, E.; Cheung, C. L.; Lieber, C. M., J. Am. Chem. Soc., 120, 8557 (1998); Hamon, M. A.; Chen, J.; Hu, H.; Chen, Y.; Itkis, M. E.; Rao, A. M.; Eklund, P. C.; Haddon, R. C., Adv. Mater., 11, 834 (1999); and Ausman, K. D.; Piner, R.; Lourie, O.; Ruoff, R. S.; Korobov, M., J. Phys. Chem. B, 104, 8911 (2000), the entire teachings of which are incorporated herein by reference.

Optionally, the nanotubules containing nitric oxide or a gas with nitric oxide-like biological activity are entrained within a polymer. These nanotubules can optionally contain oxygen or other gases as well. Nanotubules that are entrained within a polymer are distributed, preferably homogeneously, throughout the polymer composition. To become entrained within a polymer, nanotubules are typically added to a non-solidified polymer, a solution comprising a polymer, or a solution comprising monomers that are subsequently polymerized.

Polymers with nanotubules entrained therein can be hydrophilic, amphipathic, or hydrophobic, but are preferably hydrophobic. Suitable polymers include teflons (e.g., poly(tetrafluoroethylene)), polylactides, polyurethanes, polyanhydrides, and polyesters. Preferred polymers include copolymers comprising isobutylene and styrene repeat units, such as a styrene-isobutylene-styrene block copolymer. It is to be understood that not every nanotubule entrained within a polymer needs to contain nitric oxide or a gas with nitric oxide-like biological activity in order to be encompassed within the invention.

An article is a three-dimensional object or item having some useful function. An article comprises (e.g., incorporates or is coated with) nanotubules containing nitric oxide or a gas with nitric oxide-like biological activity, or a polymer with such nanotubules entrained therein. The article can be a device for which a useful result can be achieved by nitric oxide release, including a medical device suitable for implantation at a treatment site in a subject.

Articles of the present invention can also serve as exogenous sources of nitric oxide, whereby an aqueous solution is contacted with the article and the aqueous solution is administered to an individual. The aqueous solution and the article can be contacted, such as when the aqueous solution passes through or over the article, or the aqueous solution can be stored in the article for a short term (e.g., minutes or hours) or a long term (e.g., days, weeks, months, or longer). The aqueous solution can be administered or infused orally, intranasally, rectally, subcutaneously, intramuscularly, intravenously, intraurethally, intrauterinely, topically, intrabronchially, or by aerosol or spray. Aqueous solutions, after contacting such articles, can be used as a means of delivering nitric oxide or a gas with nitric oxide-like activity to an individual.

Medical devices of the present invention include devices suitable for implantation in a subject, contact with mucous membranes, or contact with biological fluids. The medical device can deliver nitric oxide to the treatment site in the subject after implantation. In one example, implanting a medical device, such as a stent, in a subject at a treatment site at risk for clot formation can be used to inhibit or prevent restenosis. Examples of suitable medical devices include medical tubing, catheters, and stents. Medical tubing, as used herein, is tubing suitable for internal use in a mammal or for contact with biological fluids. Stents of the present invention can additionally comprise one or more pharmaceutically active agents. Preferably, stents of the present invention are coated with an antiproliferative, immunosuppressive, antibiotic, and/or antimicrobial pharmaceutically active agent and a nanotubule as described herein.

A “treatment site,” as defined herein, is a site where surgery is performed or a medical device is implanted. A “treatment site” additionally includes a site where an aqueous solution is delivered or infused. Also, a “treatment site” includes a site in the body of a subject in which a desirable therapeutic effect can be achieved by contacting the site with nitric oxide or a substance having the activity of nitric oxide. “Treatment sites at risk for clot formation,” as defined herein, are sites within the circulatory system where blood clots are at risk of forming, e.g., where there is plaque formation, atherosclerosis, an injury to the blood vessel wall, or an obstruction to blood flow. In particular, the treatment sites are located next to, contiguous with, or within a vein, artery, capillary, or other blood vessel. A “subject” or “individual” refers to a human or an animal such as a veterinary animal (e.g., dogs, cats, and the like) and farm animals (e.g., horses, cows, pigs, and the like).

Treatment sites are found, for example, at sites within the body which develop restenosis, injury or thrombosis as a result of trauma caused by contacting the site with a synthetic material or a medical device. For example, restenosis can develop in blood vessels which have undergone coronary procedures or peripheral procedures with PTCA balloon catheters (e.g. percutaneous transluminal angioplasty). Restenosis is the development of scar-like tissue from about three to six months after the procedure and results in narrowing of the blood vessel. Nitric oxide and gases with the biological activity thereof reduce restenosis by inhibiting platelet deposition and smooth muscle proliferation. Nitric oxide and gases with the biological activity thereof also inhibit thrombosis by inhibiting platelets and can limit injury by serving as an anti-inflammatory agent.

A site in need of treatment with nitric oxide or gases with the biological activity thereof often develops at vascular sites which are in contact with a synthetic material or a medical device. For example, stents are often inserted into blood vessels to prevent restenosis and re-narrowing of a blood vessel after a procedure such as angioplasty. Platelet aggregation resulting in thrombus formation is a complication which can result from the insertion of stents. Nitric oxide is an antiplatelet agent and can consequently be used to lessen the risk of thrombus formation associated with the use of these medical devices. Other examples of medical devices which contact vascular sites and thereby increase the risk of thrombus formation include sheaths for veins and arteries and GORE-TEX surgical prostheses.

The need for treatment with nitric oxide and gases with the biological activity thereof can also develop at non-vascular sites, for example at sites where a useful therapeutic effect can be achieved by reducing an inflammatory response. Examples include the airway, the gastrointestinal tract, bladder, uterus and corpus cavernosum. Thus, the compositions, methods and devices of the present invention can be used to treat respiratory disorders, gastrointestinal disorders, urological dysfunction, impotence, uterine dysfunction and premature labor. NO delivery at a treatment site can also result in smooth muscle relaxation to facilitate insertion of a medical device, for example in procedures such as bronchoscopy, endoscopy, laparoscopy and cystoscopy. Delivery of NO can also be used to prevent cerebral vasospasms post hemorrhage and to treat bladder irritability, urethral strictures and biliary spasms.

The need for treatment with nitric oxide or gases with the biological activity thereof can also arise external to the body in medical devices used to treat bodily fluids temporarily removed from body for treatment, for example blood. Examples include conduit tubes within heart lung machines, tubes of a dialysis apparatus and catheters.

The method of delivering nitric oxide or gases with the biological activity thereof to a treatment site in a subject comprises implanting a medical device which comprises one or more compounds of the present invention at the treatment site. Nitric oxide or gases with the biological activity thereof can be delivered to bodily fluids, for example blood, by contacting the bodily fluid with a tube or catheter comprising one or more nanotubules of the present invention. Examples of treatment sites in a subject, medical devices suitable for implementation at the treatment sites and medical devices suitable for contacting bodily fluids such as blood are described in the paragraphs hereinabove.

“Implanting a medical device at a treatment site” refers to bringing the medical device into actual physical contact with the treatment site or, in the alternative, bringing the medical device into close enough proximity to the treatment site so that nitric oxide or gases with the biological activity thereof released from the medical device comes into physical contact with the treatment site. A bodily fluid is contacted with a medical device, e.g., a tube or catheter, when, for example, the bodily fluid is temporarily removed from the body for treatment by the medical device, and the coating is an interface between the bodily fluid and the medical device. Examples include the removal of blood for dialysis or by heart lung machines.

Optionally, articles of the present invention are coated with nanotubules or a polymer with nanotubules entrained therein. An article, for example, a medical device such as a stent, tube or catheter, can be coated with one or more compositions of the present invention. In order to form a coating, a solution comprising a composition containing nitric oxide or a gas with nitric oxide-like biological activity is contacted with an article insoluble in the solution. When the composition is insoluble in solution, the composition precipitates from the solution and coats the article. When the composition is soluble in the solution, the article can be dipped into or sprayed with the solution and then dried in vacuo or under a stream of an inert gas such as nitrogen or argon, thereby coating the article.

Articles of the present invention also include condoms. Condoms can be designed for use by either males or females. Condoms can be formed from suitable materials, particularly polymers. Suitable materials include latex, rubber, and polyurethane. The nanotubules can be entrained in the condom, particularly when the condom is comprised of one or more polymers, or can coat the condom.

Articles of the present invention also include pills and capsules comprising a pharmaceutically active agent and a coating or shell comprising one or more nanotubules containing nitric oxide or a gas with nitric oxide-like biological activity. Coated tablets of the invention can be prepared by a method comprising the step of contacting a tablet core comprising a pharmaceutically active agent with a coating solution comprising a solvent, at least one coating agent dissolved or suspended in the solvent, one or more nanotubules, and, optionally, one or more plasticizing agents. Preferably, the solvent is an aqueous solvent, such as water or an aqueous buffer, or a mixed aqueous/organic solvent. Suitable coating agents include beeswax, glyceryl monostearate, shellac, cetyl alcohol, mastic, stearic acid, cellulose, ethyl cellulose, hydroxypropylmethylcellulose, hydroxypropylmethylcellulose phthalate polymer, hydroxypropylcellulose, cross-linked sodium carboxymethylcellulose, microcrystalline cellulose, ethylcellulose, methylcellulose, cellulose acetophthalate, methylcellulose acetophthalate, cellulose acetate tetrahydrophalate, cellulose acetopropionate, cellulose trimetallate, cellulose acetate, cellulose butyrate, carboxymethyl starch, starches, starch derivates, polyvinyl acetate, carboxyvinylpolymers, polyvinylalcohol optionally cross-linked with glyoxal, formaldehyde, or glutaraldehyde, cross-linked polyvinylpyrrolidone, poly(methyl vinyl ethers-co-maleic anhydride), neutral copolymers of polymethacrylic acid esters (Eudragit L30D), copolymers of methacrylic acid and methacrylic acid methyl ester (Eudragits), a neutral copolymer of polymethacrylic acid esters containing metallic stearates, potassium methacrylate-divinylbenzene copolymer, acrylic and methacrylic copolymer, methyl methacrylate, methacrylic acid, ethyl acetate latexes, beta-cyclodextrine, dextrine derivatives, mannitol, lactose, sorbitol, xylitol, glucans, scleroglucans, mannans, galactomannans, carrageenan and derivatives thereof, xanthans, alginic acid and derivatives thereof, pectin, amylose, sandarac gum, and mixtures thereof. Suitable plasticizers include polyethylene glycol (PEG 200, PEG 1000), polyoxyethylene glycols, diethyl phthalate, dibutyl phthalate, triacetin, monoglyceride, rape seed oil, olive oil, sesame oil, acetyltributylcitrate, acetyltriethylcitrate, glycerin, sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, hydrogenated castor oil, fatty acids, substituted glycerides and triglycerides, glycerol, D-sorbitol, sucrose, mannitol, fructose, sugar alcohol, isomerized sugars, and propylene glycol. Typically, the tablet core is contacted with the coating solution until the weight of the tablet core has increased by an amount ranging from about 1% to about 20%, indicating the deposition of a suitable coating on the tablet core to form a coated tablet.

Capsules typically comprise a shell and a solid or liquid core comprising a pharmaceutically active agent. The shell can be hard or soft and is typically comprised of a suitable solid coating material, such as gelatin, agar, sodium alginate, pectin, carageenan, carboxymethyl cellulose, gelant gum, poly(sodium acrylate), poly(sodium methacrylate), hydroxypropylmethylcellulose, hydroxyethylcellulose, hydroxypropylcellulose, polyvinylpyrrolidone, polyvinyl alcohol, acrylic acid polymer, methacrylic acid copolymers, ethyl acrylate-methyl methacrylate copolymers, and mixtures thereof; one or more nanotubules; and a plasticizer or another suitable material to modify the properties of the shell, such as those named above. The capsules can contain the pharmaceutically active agents in admixture with filler such as lactose, binders such as starches, and/or lubricants such as talc or magnesium stearate and, optionally, stabilizers such as gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens such as e.g., Tween 20 and Tween 80 (ICI Speciality Chemicals)); polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (block copolymers of ethylene oxide and propylene oxide); poloxamines (a tetrafunctional block copolymer derived from sequential addition of propylene oxide and ethylene oxide to ethylenediamine), dialkylesters of sodium sulfosuccinic acid (e.g., a dioctyl ester of sodium sulfosuccinic acid); sodium lauryl sulfate; alkyl aryl polyether sulfonate; a mixture of sucrose stearate and sucrose distearate; p-isononylphenoxypoly-(glycidol); decanoyl-N-methylglucamide; n-decyl-beta-D-glucopyranoside; n-decyl-beta-D-maltopyranoside; n-dodecyl-beta-D-glucopyranoside; n-dodecyl-beta-D-maltoside; heptanoyl-N-methylglucamide; n-heptyl-beta-D-glucopyranoside; n-heptyl-beta-D-thioglucoside; n-hexyl-beta-D-glucopyranoside; nonanoyl-N-methylglucamide; n-noyl-beta-D-glucopyranoside; octanoyl-N-methylglucamide; n-octyl-beta-D-glucopyranoside; and octyl-beta-D-thioglucopyranoside. In hard capsules, the solid core can be comprised of particles; each particle can have a coating (e.g., with a coating suitable for tablets, as described above) comprising one or more nanotubules of the present invention. In soft capsules, the active compounds can be dissolved or suspended in suitable liquids, such as fatty oils, liquid paraffin, or liquid polyethylene glycols. In addition, stabilizers can be added.

Articles other than pills and capsules can also comprise a pharmaceutically active agent. Suitable pharmaceutically active agents for use in the present invention include antibiotics, antimicrobials, antiproliferative agents, immunosuppressive agents, anti-inflammatory agents and COX-2 inhibitors. Examples of antibiotics and antimicrobials include streptomycin, rifamycin, amphotericin B, griseofulvin, penicillin, cephalothin, cefazolin, chloramphenicol, fluconazole, clindamycin, erythromycin, bacitracin, vancomycin, ciprofloxacin, tetracycline, and fusidic acid. Examples of antiproliferative and immunosuppressive agents include corticosteroids, cyclosporine, tacrolimus, interferons (e.g., IFNα, IFNβ, IFNγ), mycophenolate mofetil, 15-deoxyspergualin, thalidomide, azathioprene, cyclophosphamide, azacitidine, cytarabine, fluorouracil, mercaptoprine, methotrexate, thioguanine, bleomycin, etoposide, teniposide, vincristine, vinblastine, busulfan, mechlorethamine, melphalan, thiotepa, dactinomycin, daunorubicin, doxorubicin, plicamycin, mitomycin, cisplatin, and nitrosoureas. Preferred antiproliferative and immunosuppressive agents include paclitaxel and rapamycin. Examples of anti-inflammatory agents and COX-2 inhibitors include aspirin, acetominaphen, and non-steroidal anti-inflammatory drugs (e.g., ibuprofen, naproxen, nabumetone, apazone, diclofenac, diflunisal, etodolac, fenoprofen, flurbiprofen, indomethacin, keoprofen, ketorolac, meclofenamate, oxaprozin, piroxicam, sulindac, tolmetin, rofecoxib, celecoxib, valdecoxib, meloxicam).

As used herein, a “surfactant” is an agent which preferentially absorbs to an interface between two immiscible phases, such as the interface between water and an organic polymer solution, a water/air interface or organic solvent/air interface. Surfactants generally possess a hydrophilic moiety and a lipophilic moiety. Suitable surfactants include but are not limited to phospholipids such as 1,2-Dipalmitoyl-sn-glycero-3-phosphocholine, 1,2-Distearoyl-sn-glycero-3-phosphocholine, phosphatidyl ethanolamine, phosphatidylcholine, phosphatidylethnolamine, phosphatydilinositol, phosphatidylserine, and phosphatidylglycerol; hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); tyloxapol; alcohol ethyoxylates; alkylphenol ethoxylates; fatty amine oxides; alkalonamides; ethylene oxide/propylene oxide block copolymers; poly-oxyalkylene glycols; polyoxypropylene glycol monoalkylethers; poly-(oxyethylene oxypropylene) glycol monoalkylethers; imidazolines; betaines; alkylbenzene sulfonic acid; sodium lauryl ether sulfate; alpha olefin sulfonates; phosphate esters; and sodium sulfosuccinates.

Perfluorocarbons (PFCs) are hydrocarbons with all of the hydrogen atoms replaced by fluorine, although one to five of the fluorine atoms can be another halogen. Perfluorocarbons includes perfluorodecaline, perfluorotripropylamine, perfluorooctyl bromide, and perfluorodichlorooctane.

Cyclodextrins include α-cyclodextrin, β-cyclodextrin, and γ-cyclodextrin. Cyclodextrins can be converted to polythiolated cyclodextrins, for example, by the methods disclosed in Gaddell and Defaye, Angew. Chem. Int. Ed. Engl. 30: 78, 1991 and Rojas et al., J. Am. Chem. Soc. 117: 336, 1995, the teachings of which are incorporated herein by reference. An excess of thiolating reagent can be used to form perthiolated cyclodextrins, whereby all primary alcohols are converted to thiol groups.

In the preparation of nanotubules containing nitric oxide or a gas with nitric oxide-like biological activity, the nanotubules are preferably contacted with a gas consisting essentially of nitric oxide or a gas with nitric oxide-like biological activity. The nanotubules are in contact with the gas for a sufficient amount of time to obtain a nanotubule with the desired weight percent content of the gas. More preferably, the nanotubules are contacted with an oxygen-free inert gas or combination of inert gases prior to contacting the nanotubules with nitric oxide. Examples of inert gases include nitrogen, argon, helium, and neon.

While this invention has been particularly shown and described with references to preferred embodiments thereof, it will be understood by those skilled in the art that various changes in form and details may be made therein without departing from the scope of the invention encompassed by the appended claims. The invention will now be further and specifically described by the following non-limiting Examples.

### EXAMPLES

Preparation and Assays of NO-Loaded Carbon Nanotubes (CNs)

### Example 1

### Loading and Heat Assay

A 125 mL bottle with a poly(tetrafluoroethylene)-faced (PTFE-faced), silicone rubber open-top cap was filled with glass vials and glass wool to an extent that a 2 mL vial could be placed very nearly at the top of the bottle. Single-walled carbon nanotublules (hereinafter “CN”, Aldrich 519308, CarboLex AP-grade, 17.3 mg) were placed in a 2 mL vial, which was put into the 125 mL bottle. By means of a 6 inch needle, argon gas was blown slowly through the bottom of the 125 mL bottle for 25 minutes, with egress through a hypodermic needle at the top. By the same process, NO gas was blown through the bottom of the 125 mL bottle for 20 minutes; the NO gas was first blown through granular KOH and a water bubbler to remove trace NO₂. The sealed bottle was stored in the dark at 25° C. for 7.5 hours. By means of a 6 inch needle, nitrogen gas was blown rapidly through the bottom of the 125 mL bottle for 13 minutes, with egress through a hypodermic needle at the top. The bottle was opened to atmosphere, and the 2 mL vial was removed. Deionized water (1000 μL) was added to the vial, the head space was filled with oxygen, and the vial was capped with a PTFE-faced, silicone rubber open-top cap. The cap was secured to the vial with autoclave tape, and the vial was stored at 80-90° C. for 14 hours. The vial was cooled to 25° C. A 7.5 μL aliquot of the water was found to contain 61.7 mmol nitrogen oxides (NOₓ) by chemiluminescence, corresponding to 476 nmol NO per milligram of CN, roughly 1.4% loading (w/w). A control sample of CN (33.7 mg) that was not treated with NO gas had no measurable NOₓ. A control sample of pure carbon (Aldrich 484164, glassy, spherical powder, 2-12 micron, 66.2 mg) that was treated with NO as described above had no measurable NOₓ.

### Example 2

### Bioassay (Rabbit Aortal Assay)

The capacity of a compound or composition to cause relaxation of vascular smooth muscle, measured by the degree and duration of vasodilation resulting from exposure of a blood vessel to the compound, is a measure of its ability to deliver NO in vivo. Methods reported in Jia, L., et al., Nature, 380: 221-226, 1996; Stamler, J. S., et al., Science, 276: 2034-2037, 1997; Stamler et al., Proc. Natl. Acad. Sci. USA 89: 444, 1992; Osborne et al., J. Clin. Invest. 83: 465, 1989; and the chapter by Furchgott in Methods in Nitric Oxide Research, edited by Feelisch and Stamler, John Wiley & Sons (1996), were used to measure vascular smooth muscle contraction.

By the means described in Example 1, NO-loaded CNs were prepared from 19.4 mg CN, with, sequentially, 25 minutes of Ar gas flow, 35 minutes of NO gas flow, and 16 hours of storage under NO. NO was removed from the 125 mL bottle by flushing with Ar gas for 25 minutes; the NO-loaded CNs were then stored under Ar for 30 hours.

New Zealand White female rabbits weighing 3-4 kg were anesthetized with sodium pentobarbital (30 mg/kg). Descending thoracic aorta were isolated, the vessels were cleaned of adherent tissue, and the endothelium was removed by gentle rubbing with a cotton-tipped applicator inserted into the lumen. The vessels were cut into 5-mm rings and mounted on stirrups in 20 mL organ baths. The rings were suspended under a resting force of 1 g in 7 ml of oxygenated Kreb's buffer (pH 7.5) at 37° C. and allowed to equilibrate for one hour. Isometric contractions were measured on a Model 7 oscillograph recorder connected to transducers (model TO3C, Grass Instruments, Quincy, Mass.). Fresh Krebs solution was added to the bath periodically during the equilibration period and after each test response. Sustained contractions were induced with 7 μM norepinephrine prior to the addition of the test compound. The assay demonstrated bioactivity; very small (approximately 160 μg) additions of NO-loaded CN to the aortal rings showed both short- and long-term relaxation. Similar amounts of CN not treated with nitric oxide had little or no activity.

### Example 3

### NO Release from NO-Loaded CNs into Phosphate-Buffered Saline

By the means described in Example 1, two samples of NO-loaded CNs (CN—NO) were prepared:
(1) From 19.4 mg CN, with, sequentially, 25 minutes of Ar gas flow, 35 minutes of NO gas flow, and 16 hours of storage under NO. NO was removed from the 125 mL bottle by flushing with Ar gas for 25 minutes; the CN—NO was stored under Ar for 30 hours. The CN—NO was stored in a 2 mL vial under ambient atmosphere for 3 days.
(2) From 23.1 mg CN, with, sequentially, 25 minutes of Ar gas flow, 35 minutes of NO gas flow, and 20 hours of storage under NO. NO was removed from the 125 mL bottle by flushing with Ar gas for 25 minutes; the CN—NO was stored under Ar for 20 hours. The CN—NO was stored in a 2 mL vial under ambient atmosphere for 2 days.

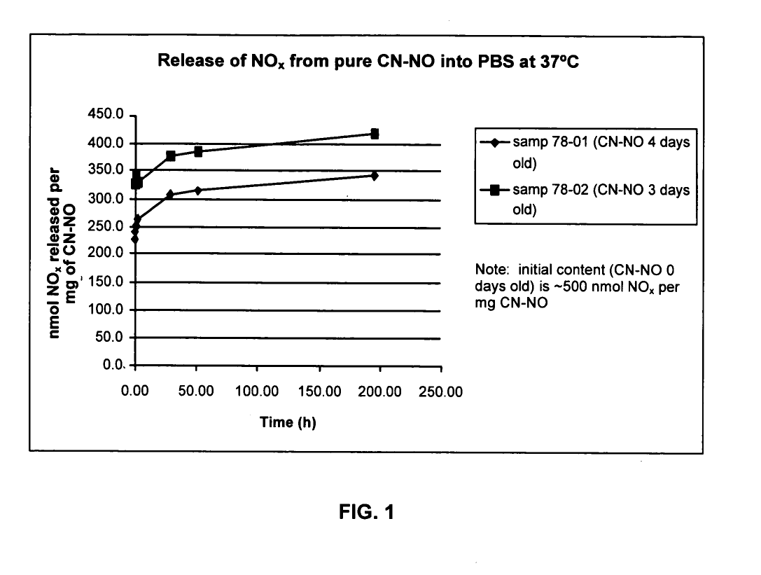

Small samples (6.0 mg of sample (1), 7.6 mg of sample (2)) were weighed into 2 mL screw-cap vials, phosphate-buffered saline (PBS, 1000 μL, 25° C.) was added at time t=0, vials were stored at 37° C., and aliquots (25 μL) were analyzed at time points for NOₓ content. Both samples showed release beyond the first day (FIG. 1).

### Example 4

### NO Release from CN—NO Entrained in SIBS (Styrene-Isobutylene-Styrene Copolymer) into PBS

By the means described in Example 1, two samples of CN—NO were prepared:
(1) From 19.4 mg CN, with, sequentially, 25 minutes of Ar gas flow, 35 minutes of NO gas flow, and 16 hours of storage under NO. NO was removed from the 125 mL bottle by flushing with Ar gas for 25 minutes; the CN—NO was stored under Ar for 30 hours. The CN—NO was stored in a 2 mL vial under ambient atmosphere for 3 days.
(2) From 23.1 mg CN, with, sequentially, 25 minutes of Ar gas flow, 35 minutes of NO gas flow, and 20 hours of storage under NO. NO was removed from the 125 mL bottle by flushing with Ar gas for 25 minutes; the CN—NO was stored under Ar for 20 hours. The CN—NO was stored in a 2 mL vial under ambient atmosphere for 2 days.

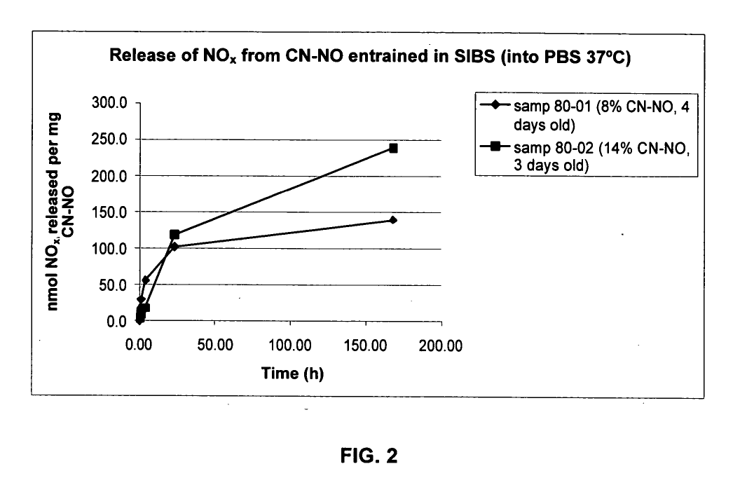

Small samples (4.8 mg of sample (1), 8.3 mg of sample (2)) were weighed into 2 mL screw-cap vials. A stock solution of SIBS polymer in dichloromethane (5.5058 g in 100 mL) was prepared and bubbled with argon for 15 min; 1 mL was added to each vial (approximately 52.2 mg SIBS). Solvent was removed by blowing nitrogen through each vial (approxmately 5 minutes) to give CN—NO entrained in SIBS. Samples were stored in the dark at 25° C. for 24 hours. PBS (1500 μL, 37° C.) was added at time t=0, vials were stored at 37° C., and aliquots (25 μL) were analyzed (described earlier) at time points for NOₓ content. Both samples showed sustained release beyond the first day (FIG. 2).

### Example 5

### Loading of CN Entrained in SIBS Polymer

Single-walled CNs (Aldrich 519308, CarboLex AP-grade, 16.0 mg) were placed in a 2-mL vial. A stock solution of SIBS polymer in dichloromethane (5.5058 g in 100 mL) was prepared and bubbled with argon for 15 minutes; 1 mL was added to the vial (approximately 52.2 mg SIBS). Solvent was removed by blowing nitrogen through the vial (approximately 5 minutes) to give CN entrained in SIBS. The vial was treated with NO as described in Example 1, with 25 minutes of Ar gas flow, 31 minutes of NO gas flow, and 24 hours of storage under NO. NO was removed from the 125 mL bottle by flushing with Ar gas for 36 minutes. The vial containing CN—NO in SIBS was briefly exposed to ambient atmosphere while it was removed from the 125 mL bottle; the vial was capped for 4.5 hours. Deionized water (1000 μL) was added to the vial, the head space was filled with oxygen, and the vial was capped with a PTFE-faced, silicone rubber open-top cap. The cap was secured to the vial with autoclave tape, and the vial was stored at 80-90° C. for 12 hours. The vial was cooled to 25° C. After 6 hours from the time of cooling, a 10-μL aliquot of the water was found to contain 18.4 nmol NOₓ, corresponding to 115 nmol NO per milligram of CN. After 144 hours from the time of cooling, a 10-μL aliquot of the water was found to contain 22.6 nmol NOₓ, corresponding to 141 nmol NO per milligram of CN.

While this invention has been particularly shown and described with references to preferred embodiments thereof, it will be understood by those skilled in the art that various changes in form and details may be made therein without departing from the scope of the invention encompassed by the appended claims.

1. A composition comprising a compound that non-covalently binds nitric oxide or a gas with nitric oxide-like biological activity and nitric oxide or a gas with nitric oxide-like activity non-covalently bound to said compound.

2. A nanotubule, wherein said nanotubule contains nitric oxide or a gas with nitric oxide-like biological activity and wherein the interior of said nanotubule is substantially free of oxygen.

3. The nanotubule of claim 2, wherein the nanotubule contains a gas with nitric oxide-like biological activity.

4. The nanotubule of claim 3, wherein the gas with nitric oxide-like biological activity is nitrogen dioxide, dinitrogen trioxide, an alkyl nitrite, or ethyl nitrite.

5. The nanotubule of claim 2, wherein the nanotubule contains nitric oxide.

6. The nanotubule of claim 2, wherein the nanotubule has a diameter between about 1 nm about 50 nm and a length between about 10 nm and about 100 μm.

7. A nanotubule, wherein said nanotubule is functionalized with a functional group and contains nitric oxide or a gas with nitric oxide-like biological activity.

8. The nanotubule of claim 7, wherein the nanotubule contains nitric oxide.

9. The nanotubule of claim 8, wherein said nanotubule is functionalized with fluoride, an alcohol, an amine, an alkyl group, or a combination thereof.

10. A nanotubule, wherein the ends of said nanotubule are functionalized with one or more capping molecules and wherein said nanotubule contains nitric oxide or a gas with nitric oxide-like biological activity.

11. The nanotubule of claim 10, wherein said capping molecule is attached to a nanotubule by one or more amide, ester, carbonate, carbamate, urea, acylurea, phosphate ester, phosphonate ester, sulfonate ester, or sulfate ester moieties, or a combination thereof.

12. The nanotubule of claim 2, wherein the nanotubule is single-walled.

13. The nanotubule of claim 2, wherein the nanotubule is multi-walled.

14. The nanotubule of claim 2, wherein the nanotubule is open-ended.

15. The nanotubule of claim 2, wherein nitric oxide or the gas with nitric oxide-like biological activity contained by the nanotubule comprises 0.5-10 weight percent of said nanotubule.

16. The nanotubule of claim 15, wherein nitric gas or the gas with nitric oxide-like biological activity contained by the nanotubule comprises 0.5-6 weight percent of the nanotubule.

17. An article comprising one or more nanotubules containing nitric oxide or a gas with nitric oxide-like biological activity.

18. The article of claim 17, wherein said article is a bag containing intravenous fluid, a syringe, or medical tubing.

19. The article of claim 17, wherein the nanotubules contain nitric oxide.

20. The article of claim 19, further comprising a polymer with the nanotubules entrained therein.

21. The article of claim 20, wherein the polymer coats the article.

22. The article of claim 19, wherein the polymer is a copolymer comprising isobutylene and styrene repeat units.

23. The article of claim 19, wherein the polymer is poly(tetrafluoroethylene).

24. The article of claim 19, further comprising a pharmaceutically active agent.

25. The article of claim 19, wherein the article is a condom.

26. A pill or capsule comprising a pharmaceutically active agent and a coating comprising one or more nanotubules containing nitric oxide or a gas with nitric oxide-like biological activity.

27. The pill or capsule of claim 26, wherein the pharmaceutically active agent is an antiproliferative agent.

28. The pill or capsule of claim 27, wherein the antiproliferative agent is paclitaxel or rapamycin.

29. The pill or capsule of claim 26, wherein the pharmaceutically active agent is a COX-2 inhibitor.

30. The pill or capsule of claim 29, wherein the COX-2 inhibitor is aspirin or a non-steroidal anti-inflammatory drug.

31. A medical device suitable for implantation in a subject, for contact with mucous membranes, or for contact with a biological fluid, wherein said device comprises one or more nanotubules containing nitric oxide or a gas with nitric oxide-like biological activity.

32. The medical device of claim 31, wherein said device is medical tubing or a stent.

33. The medical device of claim 32, wherein said device is a stent comprising an antiproliferative or immunosuppressive pharmaceutically active agent.

34. The medical device of claim 33, wherein the pharmaceutically active agent is paclitaxel or rapamycin.

35. A method of inhibiting restenosis, comprising implanting a stent comprising one or more nanotubules containing nitric oxide or a gas with nitric oxide-like biological activity.

36. The method of claim 35, wherein the nanotubules contain nitric oxide.

37. The method of claim 36, wherein the stent is coated with a pharmaceutically active agent having antiproliferative or immunosuppressive activity.

38. The method of claim 37, wherein the pharmaceutically active agent is paclitaxel or rapamycin.

39. A method of delivering nitric oxide to a treatment site by implanting a medical device comprising one or more nanotubules containing nitric oxide or a gas with nitric oxide-like biological activity.

40. The method of claim 39, wherein the nanotubules contain nitric oxide.

41. The method of claim 40, wherein the treatment site is at risk for clot formation.

42. A method of preparing nanotubules containing nitric oxide or a gas with nitric oxide-like biological activity, comprising the step of contacting said nanotubules with nitric oxide or a gas with nitric oxide-like biological activity, wherein nitric oxide or the gas with nitric oxide-like biological activity is substantially free of oxygen.

43. The method of claim 42, wherein the nanotubules are contacted with a gas consisting essentially of nitric oxide.

44. The method of claim 43, further comprising contacting the nanotubules with an oxygen-free inert gas or combination of inert gases prior to contacting the nanotubules with nitric oxide.

45. A polymer entrained with nanotubules, wherein said nanotubules contain nitric oxide or a gas with nitric oxide-like biological properties.

46. The polymer of claim 45, wherein said nanotubules contain nitric oxide.

47. The polymer of claim 46, wherein said polymer is a copolymer comprising isobutylene and polystyrene repeat units.

48. A composition comprising a polymer and nanotubules entrained in the polymer, wherein said nanotubules contain nitric oxide or a gas with nitric oxide-like biological activity.

49. The composition of claim 48, wherein the nanotubules contain nitric oxide.

50. A method of administering nitric oxide or a gas with nitric oxide-like properties to an individual, comprising the step of contacting an aqueous solution with an article comprising one or more nanotubules containing nitric oxide or a gas with nitric oxide-like biological activity and administering said aqueous solution to said individual.

51. The method of claim 50, wherein the article is a bag containing intravenous fluid, a syringe, or medically-suitable tubing.
